(19) United States
(12) Patent Application Publication
(10) Pub. No.: US 2016/0168619 A1
(43) Pub. Date: Jun. 16, 2016

(54) COLORIMETRIC ASSAY FOR L-GLUTAMINE AND RELATED ASSAY KIT

(71) Applicant: Shufang Tao, North Logan, UT (US)

(72) Inventor: SHUFANG TAO, NORTH LOGAN, UT (US)

(73) Assignee: SHUFANG TAO, NORTH LOGAN, UT (US)

(21) Appl. No.: 14/802,041

(22) Filed: Jul. 17, 2015

(30) Foreign Application Priority Data
Dec. 11, 2014 (CN) 201410775824.6

(51) Int. Cl. C12Q 1/527

(52) U.S. Cl. CPC C12Q 1/527; G01N 2333/988

(57) ABSTRACT

The present invention is comprised of a novel assay and related kit for analyzing the L-glutamine content in samples such as cell cultures and blood serum, plasma, urine, cell, tissue samples. This enzymatic reaction is highly specific to L-glutamine and the enzyme converts L-glutamine to the blue compound indigoidine which is visible and can be accurately measured using a spectrophotometer at 600 nm. This single-enzyme assay provides a quick and accurate assay for determination of the L-glutamine concentration. A kit is designed and described based on this L-glutamine assay.

### BACKGROUND

L-glutamine is a naturally occurring amino acid in dietary protein. It is a molecule that plays an essential role in amino acid biosynthesis and metabolism as well as many other important metabolic pathways. It is used as one of the twenty proteinogenic amino acids in protein synthesis, a major energy source, and an amino donor. Glutamine can directly cross the blood-brain barrier.¹ It not only circulates in the blood, but also can be stored in the skeletal muscles. Deficiency in glutamine is associated with a number of diseases. Thus, the concentration of L-glutamine is an indication of several diseases. An efficient and accurate assay for determining L-glutamine concentration is useful for diagnosis of these diseases. Additionally, given the fact that glutamine is an important nutrient and key metabolites for cell cultures, it is an important parameter to understand the cell metabolism and optimize the cell growth. Current bioassays are based on a two-enzyme system.² For example, in a UV-method, L-glutamine is first converted to L-glutamate by glutaminase, and then L-glutamate is dehydrogenated by glutamic dehydrogenase with nicotinamide adenine dinucleotide⁺ (NAD⁺) as a cofactor to yield α-ketoglutarate, NH⁴⁺ and NADH. The formation of NADH is measured at 340 nm to calculate the amount of L-glutamate and thus the original glutamine.^{3,4} However, compounds with UV absorptions at 340 nm in the samples may interfere with the determination of the formed NADH. Alternatively, this method can be modified by adding 3-(4,5-dimethylthiazol-2-yl)-2,5-diphenyltetrazolium bromide into the reaction system so that the product can be measured at 565 nm.⁵

The disadvantages of these double-enzymatic assays include the involvement of two enzymes and the interference of the possibly existing L-glutamate in the samples. The more enzymes involved in the process, the more reaction components and buffers are used, and the larger possibility to gain errors. It is very important to ensure the conversions of the substrates in these reactions are complete. If a sample contains L-glutamate, the amount of endogenous L-glutamate must be determined and subtracted from the total L-glutamate amount after deamination of L-glutamine.

In this invention, we developed a new assay for analyzing L-glutamine concentration using an indigoidine synthetase. Indigoidine is natural blue pigment produced by bacteria such as Streptomyces. This compound is synthesized by a nonribosomal peptide synthetase from two molecules of L-glutamine (FIG. 1). Thus, quantification of the production of this blue pigment will allow the determination of the concentration of L-glutamine. The molar ratio of indigoidine to L-glutamine is 1:2. The biosynthesis of indigoidine has been well studied and several indigoidine synthetases have been identified from different microorganisms, such as IndC from Erwinia chrysanthemi,⁶ BpsA from Streptomyces lavendulae⁷ and Streptomyces aureofaciens,⁸ and Sc-IndC from Streptomyces chromofuscus.⁹ In this work, we established an assay that consists of an indigoidine synthetase, adenosine triphosphate (ATP), Mg²⁺ and L-glutamine. The formation of the blue product is monitored at 600 nm (FIG. 2). A standard curve is established to correlating the absorbance of a reaction mixture at 600 nm to the amount of L-glutamine in the sample.

### SUMMARY

A novel assay for analyzing the L-glutamine content in samples such as cell cultures and blood serum, plasma, urine, cell, tissue samples. This enzymatic reaction is highly specific to L-glutamine and the enzyme converts L-glutamine into a blue compound indigoidine which is visible, and can be accurately measured using a spectrophotometer at 600 nm (FIG. 1). This single-enzyme assay provides a quick and accurate assay for determination of the L-glutamine concentration.

### BRIEF DESCRIPTION OF THE DRAWINGS

FIG. 1 illustrates the assay of L-glutamime through its conversion into a blue pigment by an indigoidine synthetase.

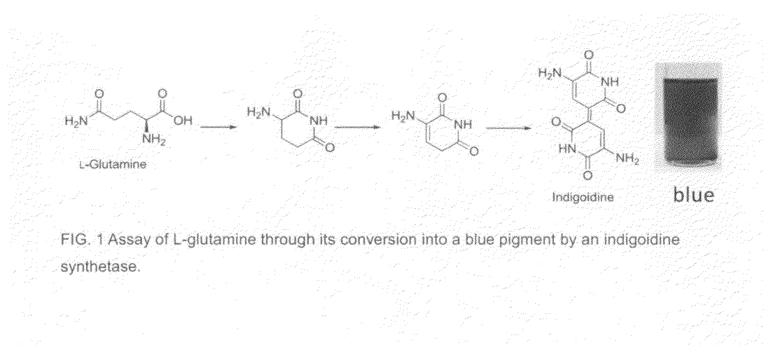

FIG. 2 is the UV spectrum of the blue pigment indigoidine.

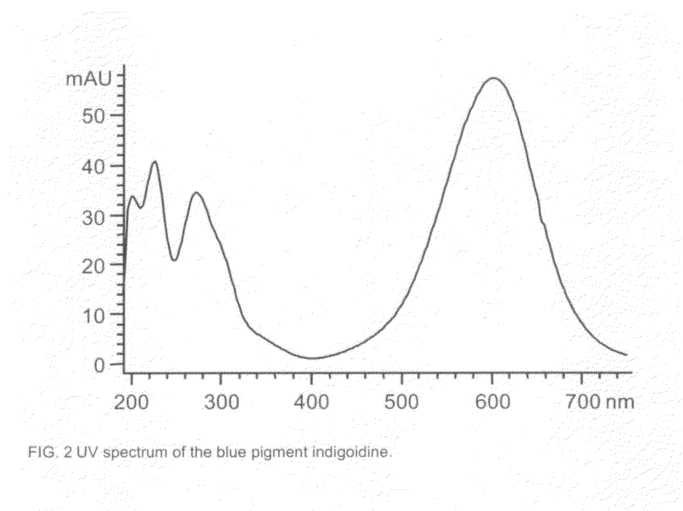

FIG. 3 is a standard curve for determination of L-glutamine concentration on a UV-vis spectrophotometer.

FIG. 4 is a standard curve for determination of L-glutamine concentration on a 96-well plate reader.

### DETAILED DESCRIPTION

The present disclosure covers methods for analyzing the concentration of L-glutamine in various samples. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

In one embodiment, the present disclosure provides methods for analysis of L-glutamine content. By way of example, the present disclosure provides for analyzing L-glutamine content through an indigoidine synthetase BpsA from Streptomyces lavendulae. The methods described herein generally provide for a novel visible assay for measurement of L-glutamine content that is different from the existing conventional two-enzyme methods. Preferably, this assay uses a single indigoidine synthetase to convert L-glutamine into a visible compound that can be accurately quantified. The details for this assay are provided.

The present disclosure also provides for the methods to produce the enzyme used in this assay.

The genes encoding an indigoidine synthetase such as BpsA can be directly amplified from the genome or cDNA of a related microbial strain or chemically synthesized. The genes may be modified for, improved activity or expression.

Expression of an indigoidine synthetase can be achieved in Escherichia coli, Streptomyces coelicolor, Streptomyces lividans or other microbial strains. Any suitable bacterial strain, vector or culture condition may be used for the production of the indigoidine synthetase. By way of example, suitable bacterial strains include E. coli strains. Alternatively, any species or strain of Streptomyces may be used. Broadly, a suitable microbial strain is any strain capable of expressing an indigoidine synthetase. There is no requirement that the mere expression of an indigoidine synthetase in a suitable microbial strain result in the production of the enzyme. In some embodiments, an indigoidine synthetase may be generated by a vector or vectors that encode for the proteins. The vector or vectors may be plasmids.

The enzyme may be engineered to contain a tag for protein purification, such as a C-terminal or/and N-terminal hexahistdine-tag. The enzyme can be purified through any or combinations of chromatography methods such as affinity and gel filtration chromatography methods.

The enzyme can be activated by a 4′-phosphopantetheine transferase (PPTase) in the expression host or through in vitro reactions.

To measure the amount of L-glutamine, the sample is reacted with an indigoidine synthetase, ATP, and Mg²⁺ in an appropriate buffer at a suitable temperature for suitable time. The formation of the product indigoidine is measured based on the absorption of the reaction mixture at a wavelength such as 600 nm. A standard curve that correlates the absorbance of the product to the amount of L-glutamine is first established using pure L-glutamine. The amount of L-glutamine in a specific sample can be calculated based on the measured absorbance data and the standard curve.

The following examples are illustrative only and are not intended to limit the disclosure in any way. One skilled in the art would recognize various known methods and conditions for expressing an indigoidine synthetase, for purifying the protein, and carrying out the indigoidine synthesis reaction to measure the amounts of blue product and L-glutamine. Each of these various embodiments are within the scope of the invention.

### EXAMPLES

The following material and methods may be used in carrying out the various embodiments of the invention.

Example 1. Bacterial strains, vectors, and culture conditions

Streptomyces lavendulae ATCC 11924 was obtained from the American Type Culture Collection (ATCC). It was grown at 30° C. in YEME medium (yeast extract 3 g/l; peptone 5 g/l; malt extract 3 g/I, and glucose 10 g/I) for the preparation of genomic DNA. E. coli XL1-Blue was purchased from Agilent and E. coli BAP1 was obtained from Stanford University.

E. coli XL1-Blue (Agilent) and pJET1.2 (Fermentas) were used for DNA cloning and sequencing. E. coli BAP1 and pET28a (Novagen) were used for protein expression. E. coli cells were grown in Luria-Bertani (LB) medium. When necessary, appropriate antibiotics were added at the following concentrations: ampicillin, 50 μg/ml; and kanamycin, 50 μg/ml. For protein expression, 200 μM of isopropyl-beta-D-thiogalactopyranoside (IPTG) was added into the E. coli BAP1 cultures for induction.

### Example 2. DNA manipulations

The genomic DNA of S. lavendulae ATCC 11924 was isolated using standard methods. Plasmids in E. coli were extracted using a GeneJET™ Plasmid Miniprep Kit (Fermentas).

### Example 3. Expression of BpsA in E. coli BAP1

The 3.9-kb gene bpsA was amplified by PCR from the genome of S. lavendulae ATCC 11924 with Phusion® Hot Start High-Fidelity DNA Polymerase (New England Biolabs) using a pair of primers, 5′-aaCATATGactcttcaggagaccagcgtgctc-3′ (the Ndel site is bolded) and 5′-atAAGCTctcgccgagcaggtagcggatgtg-3′ (the HindIII site is bolded). The amplified bpsA was ligated into the cloning vector pJET1.2 to yield pJET1.2-bpsA for sequencing.

The bpsA insert was excised from pJET1.2-bpsA with Ndel and HindIII and ligated into pET28a between the same sites to generate pET28a-bpsA (Table 1). The plasmid was introduced into E. coli BAP1 and correct transformants were selected on LB agar supplemented with 50 μg/ml kanamycin. To reconstitute the biosynthesis of indigoidine, the correct transformant was grown in LB broth supplemented with 50 μg/ml kanamycin at 37° C. with shaking at 250 rpm. When the OD₆₀₀ reached 0.4-1.0, 200 μM of IPTG was added to induce the expression of BpsA at a lower temperature (18° C. or 25° C.) and the culture was maintained under the same conditions for an additional 14 hours.

### Example 4. SDS-PAGE analysis of protein expression

The induced culture of the engineered E. coli BAP1 strain from Example 3 was analyzed for protein expression by SDS-PAGE. The cells from 50 ml of culture were collected by centrifugation at 2,700×g for 5 minutes and resuspended in 3 ml of lysis buffer (20 mM Tris-Cl, 500 mM NaCl, pH 7.9). After 10 minutes of ultrasonication (18 W, 30 s of interval), the resultant lysates were centrifuged at 21,000×g for 10 minutes. Insoluble proteins were dissolved in 8 M urea. Both soluble and insoluble fractions were analyzed by 12% SDS-PAGE.

Example 5. Purification of BpsA from E. coli BAP1/pET28a-bpsA

Plasmid pET28a-bpsA was introduced to E. coli BAP1. The strain was grown in 1l of LB medium supplemented with 50 μg/ml kanamycin at 37° C. with shaking at 250 rpm. When the OD₆₀₀ reached 0.6, 200 μM (final concentration) of IPTG was added to induce the expression of BpsA at 18° C. The induced culture was maintained at 18° C. under the same conditions for an additional 14 hours.

The E. coli culture was harvested by centrifugation at 2,700×g for 5 minutes. The cells were resuspended in lysis buffer (20 mM Tris-Cl, 500 mM NaCl, pH 7.9) and lysed by sonication on ice. Cell debris was removed by centrifugation at 21,000×g for 30 minutes. The resultant supernatant was incubated with Ni-NTA resin for 4 hours and the mixture was loaded onto an open column, which was eluted with increased concentrations of imidazole (0.10 mM, 50 mM and 250 mM) in buffer A (50 mM Tris-HCl, pH 7.9, 2 mM EDTA, 2 mM DTT). The fractions containing BpsA were concentrated and buffer exchanged into 50 mM sodium phosphate buffer (pH 7.8) using an Amicon Ultra-15 centrifugal filter unit with Ultracel-10 membrane.

Purified BpsA was stored at −20° C. after adding 50% glycerol.

Alternatively, the purified enzyme in the sodium phosphate buffer can be freeze-dried for storage and future use.

### Example 6. Establishment of a standard curve for determination of L-glutamine concentration on a UV-Vis spectrophotometer

146.15 mg of L-glutamine was dissolved in 10 ml of 50 mM sodium phosphate buffer to make a 100 mM L-glutamine concentration. 9.52 mg of MgCl₂ and 55.12 mg of ATP were dissolved in 1 ml of 50 mM sodium phosphate buffer.

Dilute the 100 mM L-Glutamine solution with 50 mM sodium phosphate buffer into the following concentrations: 30 mM, 20 mM, 10 mM, 8 mM and 4 mM.

Each cuvette contained a 750-μl reaction system, which consisted of 7.5 μl of 1 mM ATP/MgCl₂, 37.5 μl of a L-glutamine solution, 1 μM BpsA, and 50 mM sodium phosphate buffer. To do the assay, a master mixture of 50 mM sodium phosphate buffer, BpsA, ATP and MgCl₂ was first prepared. Distribute 712.5 μl of this solution into the cuvettes. Add 37.5 μl of a L-glutamine solution into the cuvettes to make 750-μl reactions. A reaction without L-glutamine but blank sodium phosphate buffer was used as negative control. All the reactions were run in triplicate.

The cuvettes were mixed by pipetting and the reactions were allowed to sit at room temperature for 30 minutes. The UV absorbance of the reactions was recorded on a UV-vis spectrophotometer at 600 nm and the results are shown below:

| | Concentration in the reaction (mM) | | | | |
|---|---|---|---|---|---|
| | 1.5 | 1.0 | 0.5 | 0.4 | 0.2 |
| Absorbance at 600 nm | 0.1380 | 0.01050 | 0.0665 | 0.0585 | 0.0395 |
| (A₆₀₀) | | | | | |

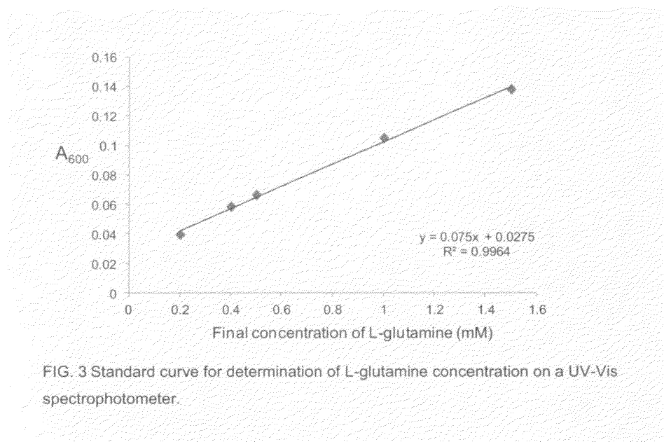

Based on these data, a standard curve that describes the relationship of A₆₀₀ values to L-glutamine concentrations were established in FIG. 3.

### Example 7. Measurement of L-glutamine in two prepared L-glutamine solutions on a UV-Vis spectrophotometer

To test the standard curve, two new L-glutamine solutions were separately prepared and added into the 750-μl reaction systems in cuvettes. The final concentrations of these reactions of L-glutamine were 1.5 mM and 1 mM, respectively. The reactions were performed in cuvettes as described in Example 6 and measured on a UV-Vis spectrophometer at 600 nm. The results from two parallel measurements are shown below:

| Actual concentration | | Calculated concentration |
|---|---|---|
| (mM) | Measured A₆₀₀ | (mM) |
| 1.5 | 0.144 | 1.55 |
| | 0.132 | 1.39 |
| Average | | 1.47 |
| 1 | 0.111 | 1.11 |
| | 0.099 | 0.95 |

| | |
|---|---|
| Average | 1.03 |

Therefore, the measured concentration of L-glutamine is close to the actual concentration of L-glutamine, confirming that this is an accurate assay for L-glutamine.

### Example 8. Measurement of L-glutamine in a microbial cell culture sample on a UV-Vis spectrophotometer

To further test this assay in cuvettes, we tested a culture of the bacterium Streptomyces chromofuscus in YM medium (0.4% glucose, 0.4% yeast extract, 1% malt extract, pH 7.3). 37.5 μl of the fermentation broth was added into three cuvettes that contained 712.5 μl of master reaction mixture prepared in Example 6 to form 750-μl reaction systems. The reactions were performed as described in Example 6, and the A₆₀₀ values of the reaction mixtures were recorded on a UV-Vis spectrophotometer at 600 nm. According to the standard curve established in Example 6, the concentration of L-glutamine in this microbial culture was determined to be 0.134±0.004 mM.

### Example 9. Establishment of a standard curve for determination of L-glutamine concentration on a 96-well plate reader

Solutions of L-glutamine, ATP and MgCl₂, and 50 mM sodium phosphate buffer were prepared as described in Example 6.

Dilute the 100 mM L-Glutamine solution with 50 mM sodium phosphate buffer into the following concentrations: 30 mM, 20 mM,10 mM, 8 mM, 4 mM, 3 mM and 2 mM.

Each well of a 96-well plate contains a 250-μl reaction system, which consisted of 2.5 μl of 1 mM ATP/MgCl₂, 12.5 μl of a L-glutamine solution, 1 μM BpsA, and 50 mM sodium phosphate buffer. To do the assay, a master mixture of 50 mM sodium phosphate buffer, BpsA, ATP and MgCl₂ was first prepared. Distribute 237.5 μl of this solution into the wells. Add 12.5 μl of a L-glutamine solution into the wells to make 250-μl reactions. The final concentrations of L-glutamine in the reactions were 1.5 mM, 1 mM, 0.5 mM, 0.4 mM, 0.2 mM, 0.15 mM and 0.1 mM. A reaction without L-glutamine but blank sodium phosphate buffer was used as negative control. All the reactions were run in triplicate.

The plate was shaken for 20 s and the reactions were allowed to sit at room temperature for 30 minutes. The UV absorbance of the reactions was recorded on a 96-well plate reader at 600 nm and the results are shown below:

| | Concentration in the reaction (mM) | | | | | | |
|---|---|---|---|---|---|---|---|
| | 1.5 | 1 | 0.5 | 0.4 | 0.2 | 0.15 | 0.1 |
| Absorbance at 600 nm (average) | 0.1052 | 0.0783 | 0.0512 | 0.0464 | 0.0317 | 0.0308 | 0.0218 |

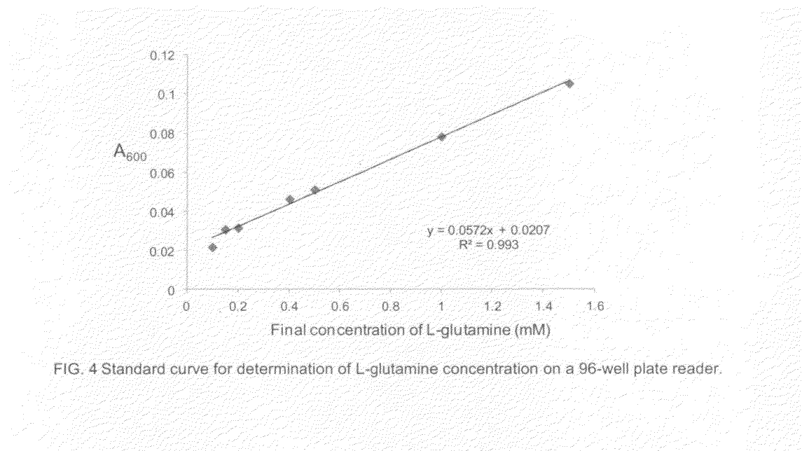

A standard curve that establishes the relationship between the absorbance at 600 nm of the reaction and L-glutamine concentration is shown in FIG. 4.

Example 10. Measurement of L-glutamine in three prepared L-glutamine solutions on a 96-well plate reader

To test the standard curve, three new L-glutamine solutions were separately prepared and added into the 250-μl reaction systems in a 96-well plate. The final concentrations of these reactions of L-glutamine were 1 mM, 0.5 mM and 0.2 mM, respectively. The reactions were performed in the 96-well plate as described in Example 9 and measured on a 96-well plate reader at 600 nm. The results are shown below:

| | Actual concentration | | Measured concentration |
|---|---|---|---|
| | (mM) | A₆₀₀ | (mM) |
| | 1 | 0.0820 | 1.07 |
| | | 0.0739 | 0.93 |
| | | 0.0789 | 1.02 |
| Average | | | 1.01 |
| | 0.5 | 0.0522 | 0.55 |
| | | 0.0488 | 0.49 |
| | | 0.0526 | 0.56 |
| Average | | | 0.53 ± 0.03 |
| | 0.2 | 0.0304 | 0.17 |
| | | 0.0324 | 0.20 |
| | | 0.0323 | 0.20 |

| | |
|---|---|
| Average | 0.19 |

The measured concentrations using this single-enzyme assay were highly consistent with the actual concentrations.

Example 11. Measurement of L-glutamine in an animal cell culture sample on a 96-well plate reader

A 3T3 mouse embryonic fibroblast cell culture was tested for the concentration of L-glutamine. 12.5 μl of the culture was taken and added into a well of a 96-well plate containing 237.5 μl of reaction components described in Example 9. Using the same method described in Example 9, the concentration of L-glutamine in this cell culture was determined to be 1.37±0.12 mM.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

### NON-PATENT CITATIONS

1. Lee, W. J.; Hawkins, R. A.; Vinã, J. R.; Peterson, D. R., Glutamine transport by the blood-brain barrier: a possible mechanism for nitrogen removal. American Journal of Physiology 1998, 274, (4 Pt 1), C1101-C1107.

2. Pye, I. F.; Stonier, C.; McGale, H. F., Double-enzymatic assay for determination of glutamine and glutamic acid in cerebrospinal fluid and plasma. Analytical Chemistry 1978, 50, (7), 951-953.

3. Lund, P., Methods of Enzymatic Analysis. VCH, Verlagsgesellschaft, Weinheim: 1986; Vol. 8, p 357-363.

4. http://www.siqmaaldrich.com/content/dam/sigma-aldrich/docs/Sigma/Bulletin/gin1bul.pdf.

5. http://www.bioassaysys.com/file dir/EGLN.pdf.

6. Reverchon, S.; Rouanet, C.; Expert, D.; Nasser, W., Characterization of indigoidine biosynthetic genes in Erwinia chrysanthemi and role of this blue pigment in pathogenicity. Journal of Bacteriology 2002, 184, (3), 654-665.

7. Takahashi, H.; Kumagai, T.; Kitani, K.; Mori, M.; Matoba, Y.; Sugiyama, M., Cloning and characterization of a Streptomyces single module type non-ribosomal peptide synthetase catalyzing a blue pigment synthesis. Journal of Biological Chemistry 2007, 282, (12), 9073-9081.

8. Novakova, R.; Odnogova, Z.; Kutas, P.; Feckova, L.; Kormanec, J., Identification and characterization of an indigoidine-like gene for a blue pigment biosynthesis in Streptomyces aureofaciens CCM 3239. Folia Microbiologica 2010, 55, (2), 119-125.

9. Yu, D.; Xu, F.; Valiente, J.; Wang, S.; Zhan, J., An indigoidine biosynthetic gene cluster from Streptomyces chromofuscus ATCC 49982 contains an unusual IndB homologue. Journal of Industrial Microbiology and Biotechnology 2013, 40, (1), 159-168.

1. A method of assaying for the concentration of L-glutamine in a given sample using a single indigoidine synthetase. The said method comprises:
(a) establishing a standard curve: reacting different concentrations of L-glutamine with an indigoidine synthetase, adenosine triphosphate and Mg²⁺ in a reaction buffer at 10-40° C. and measuring the absorption values of the reaction mixtures at 600 nm. Draw a standard curve based on the concentration of L-glutamine and the A₆₀₀ values of the reaction mixtures;
(b) mixing test samples with an indigoidine synthetase, adenosine triphosphate and Mg²⁺ in a reaction buffer under the same conditions used in (a) for the same time;
(c) measuring the absorbance of the reaction mixture at 600 nm and calculating the concentration of L-glutamine in a given sample based on the measured A₆₀₀ values and standard curve;
(d) The concentration of adenosine triphosphate in the reaction is 0.1-10 mM, the concentration of Mg²⁺ is 0.1-10 mM, pH value is 7-11, and the reaction time is 1-40 min. Before the reaction, the thiolation domain of the indigoidine synthetase needs to be activated by a phosphopantetheine transferase in vitro or in vivo.

2. The method of claim 1 further comprises a natural, recombinant, or synthesized indigoidine synthetase such as IndC, BpsA and Sc-IndC. The said IndC has an amino acid sequence shown in SEQ ID NO. 3, the said BpsA has an amino acid sequence shown in SEQ ID NO. 1 or SEQ ID NO. 4, and the said Sc-IndC has an amino acid sequence shown in SEQ ID NO. 5.

3. The method of claims 1 is a single-enzyme colorimetric assay for L-glutamine that uses a phosphate buffer.

4. The method of claims 1 is a single-enzyme colorimetric assay for L-glutamine that uses a reaction time between 1 and 40 min.

5. The method of claims 1 is a single-enzyme colorimetric assay for L-glutamine that uses a reaction temperature between 10 and 40° C.

6. A new assay kit derived from the method of claims 1 and 2 comprises indigoidine synthetase, adenosine triphosphate (0.1-10 mM), Mg²⁺ (0.1-10 mM), and a phosphate buffer with a pH within the range of 7-11.

7. The kit of claim 6 uses a single-enzyme based assay and comprises a natural, recombinant, or synthesized indigoidine synthetase such as IndC, BpsA and Sc-IndC. The said IndC has an amino acid sequence shown in SEQ ID NO. 3, the said BpsA has an amino acid sequence shown in SEQ ID NO. 1 or SEQ ID NO. 4, and the said Sc-IndC has an amino acid sequence shown in SEQ ID NO. 5.

8. The kit of claims 6 is for a single-enzyme colorimetric assay for L-glutamine that uses a phosphate buffer.

9. The kit of claims 6 is for a single-enzyme colorimetric assay for L-glutamine that uses a reaction time between 1 and 40 min.

10. The kit of claims 6 is for a single-enzyme colorimetric assay for L-glutamine that uses a reaction temperature between 10 and 40° C.

11. The method of claim 2 is a single-enzyme colorimetric assay for L-glutamine that uses a phosphate buffer.

12. The method of claim 2 is a single-enzyme colorimetric assay for L-glutamine that uses a reaction time between 1 and 40 min.

13. The method of claim 2 is a single-enzyme colorimetric assay. for L-glutamine that uses a reaction temperature between 10 and 40° C.

14. The kit of claim 7 is for a single-enzyme colorimetric assay for L-glutamine that uses a, phosphate buffer.

15. The kit of claim 7 is for a single-enzyme colorimetric assay for L-glutamine that uses a reaction time between 1 and 40 min.

16. The kit of claim 7 is for a single-enzyme colorimetric assay for L-glutamine that uses a reaction temperature between 10 and 40° C.


SEQUENCE LISTING (5 sequences)

SEQ ID NO: 1 - protein, 1282 aa, Streptomyces lavendulae
MTLQETSVLEPTLQGTTTLPGLLAQRVAEHPEAIAVAYRDDKLTFRELASRSAALADYLEHLGVSADDCVGLFVEPSIDLMVGAWGILNAGAAYLPLSPEYPEDRLRYMIENSETKIILAQQRLVSRLRELAPKDVTIVTLRESEAFVRPEGTEAPAARSARPDTLAYVIYTSGSTGKPKGVMIEHRSIVNQLGWLRETYAIDRSKVILQKTPMSFDAAQWEILSPANGATVVMGAPGVYADPEGLIETIVKHNVTTLQCVPTLLQGLIDTEKFPECVSLQQIFSGGEALSRLLAIQTTQEMPGRALINVYGPTETTINSSSFPVDPADLDEGPQSISIGSPVHGTTYHILDKETLKPVGVGEIGELYIGGIQLARGYLHRDDLTAERFLEIELEEGAEPVRLYKTGDLGQWNNDGTVQFAGRADNQVKLRGYRVELDEISLAIENHDWVRNAAVIVKNDGRTGFQNLIACIELSEKEAALMDQGNHGSHHASKKSKLQVKAQLSNPGLRDDAELAARPAFDLEGAEPTPEQRARVFARKTYRFYEGGAVTQADLLGLLGATVTAGYSRKAADLAPAELGQILRWFGQYISEERLLPKYGYASPGALYATQMYFELEGVGGLKPGYYYYQPVRHQLVLISEREATGKATAQIHFIGKKSGIEPVYKNNILEVLEIETGHMVGLFEQILPAYGLDIHDRAYEPAVKDLLDVADEDYYLGTFELVPHAGARDDQAEVYVQTHGGKVAGLPEGQYRYENGELTRFSDDIVLKKHVIAINQSVYQAASFGISVYSRAEEEWLKYITLGKKLQHLMMNGLNLGFMSSGYSSKTGNPLPASRRMDAVLGANGVDSAPMYFFVGGRISDEQIGHEGMREDSVHMRGPAELIRDDLVSFLPDYMIPNRVVVFDRLPLSANGKIDVKALAASDQVNAELVERPFVAPRTETEKEIAAVWEKALRRENASVQDDFFESGGNSLIAVGLVRELNARLGVSLPLQSVLESPTIEKLARRLEREVAQESSRFVRLHAETGKARPVICWPGLGGYPMNLRSLAGEIGLGRSFYGVQSYGINEGETPYETITEMAKKDIEALKEIQPAGPYTLWGYSFGARVAFETAYQLEQAGEKVDNLFLIAPGSPKVRAENGKVWGREASFANRGYTTILFSVFTGTISGPDLDRCLETVTDEASFAEFISELKGIDVDLARRIISVVGQTYEFEYSFHELAERTLQAPISIFKAVGDDYSFLENSSGYSAEPPTVIDLDADHYSLLREDIGELVKHIRYLLGE

SEQ ID NO: 2 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 3 - protein, 1488 aa, Dickeya chrysanthemi
MDNISNHAFNYPVLVLNKGLLPEHDDIALARYLFSALALAVSRITQNEEMIVGFHLHPQEITRWKDDECIRQYILPLNIRFNSATPIAGFIREIMTWMTPDAIHQKNAMGASVLTLGPQHALHDIFDLEISWQPPVESEPVQALTCHVASREDALVLTLRFNPARFSATQMQKLPEVWRQITASAAKNGAETLRDIGLIDDAERQRVLHAFNQTEQAWDGETTVAARLKNRAQRHPEQTAVVFRDETLSYRQLYQQAGALAHYLNALETERERCVGLFVEPSLTLMTGVWGILLSGNAYLPLSPEYPEDRLAYMLENSQTRIIVTQPHLRERLLALAPPGIQVVTSDDVDAFMRQHAHSLPDAPQNDIAPHHLAYVIYTSGSTGKPKGVMIEHHSVLNQMNWLAQTVGLNQETVILQKTPMSFDAAQWEILSPACGCRVVMGEPGVYRNPEQLVDMLAEYRVTTLQCVPTLLQALLDTERLTHCPALRQIFSGGEALQKHLAQACLETLPDCQLINLYGPTECTINNSAFRVDPVSVRQGPDTLSIGAPVANTRYYILDNCLTPVAVGQIGELYIGGDGVARGYLNRDDLTAERFIVDPFAPAGSGRRLYQTGDIASWNPDGTVQYAGRADNQVKLRGYRVELDEIRSAIETHEWVKAAAVIVRNDPFTGYQNLISFIELNAREAALMDQGNHGSHHQSKADKAQVMLQLANKGCREFPAASQPYTLDLPGKQPDEKQRLTAFSRKTYRFYDGGAVSREDILSLLHEPLLTAISRQPDALTLDELGHWLRYLGQFTSAERLLPKYTYASPGALYATQVFLELNGVAGLTAGHYYYQPVHHQLVRVSEQAAVTPGSLRLHFVGKKSAIEPIYKNNIREVLQMEMGHIIGMLDIILPDYGLGVALCDAAALDPTPLAIDLDDDYLGACDVLSGPRLPTDDDLDIYVQTAGANIADLPVGTYRYVRGDLQHIADDVIDKKHVIAINQAVYERSSFGISVASRTEGWAGYVHVGRKLQRLQMNPLNIGLMSSGYSSETGNDLPAARRFWQILGHRTGPYYFFIGGRISDEQKYSEGMREDAVHMKGPAEMIRDDLAAFMPDYMMPNKVLILDEMPLTANGKIDMKALANINVELKHKTIVAPRNPLEHQVMAIWQAKLKREEMSVDDNFFESGGNSLIAVSLINELNATLNASLPLQVLFQAPTVEKLAAWLSRARREPVSRLVQLQPKGRQAPIYCWPGLGGYCMNLRLLARQLGAERPFFGIQAHGINPDETPYATIGEMAARDIELIRQHQPHGPYTLWGYSFGARVAFETAWQLELAGEVVENLYLLAPGSPKLRDERVAAMNRKADFDNPGYLTILFSVFIGSITDPELERCLETVRDEESFVAFITGLNPALDDGLVRRITRIVAQTFEFTYTFSELQQRQLNAPVTIIKAQGDDYSFIENHGGFSAQPPTVLELMADHYSMLKAPGIDELTSVIQYQQSPPSLVG

SEQ ID NO: 4 - protein, 1283 aa, Streptomyces aureofaciens
MTLQETSVLEPTLRGTTTLPDLLAKRVAEHPEATAVAYRDEKLTYRELASRSSALAEYLRHLGVSTDDCVGLFVEPSIDLMVGAWGILSAGAAYLPLSPEYPEDRLRYMIENSQAKIILAQQRLVTRLRELAPQDVRVVTLRESEAFVLPEGQVAPAIEGARPDSLAYVIYTSGSTGKPKGVMIEHHSIVSQLGWLRETYGIDRSKTILQKTPMSFDAAQWEILSPANGATVVMGAPGVYADPEGLIETIVKYGVTTLQCVPTLLQGLLDTEKFPECTSLQQIFSGGEALSRLLAIQTTQEMPGRALINVYGPTECTINSSSYAVDPAELGEAPQSISIGAPVADTEYHILGKEDLKPVGVGEIGELYIGGGQLARGYLHRPDLTAERFLEIEVTEGAGPVRLYKTGDLGQWNPDGTVQFAGRADNQVKLRGYRVELDEISLAIENHDWVRNAAVIVKNDGRTGFQNLIACVELSEKEAALMDQGNHGSHHASKKSKLQVKAQLSNPGLRDDADLAARVAYDLPGAEPTPEQRSRVFARKTYRFYEGGAVTEADLLALLGGQVPAAYSRKAADLAPAELGQILRWFGQYLSEERLLPKYGYASPGALYATQLYFELEGVGGLQPGYYYYQPQRHQLVLISEKAATGRPTAHIHFIGKRGGIEPVYKNNIQEVLEIETGHIVGLFEQVLPAYGLDIRDLAYEPAVRDLLDVPEEDFYLGTFELVPHTGRREDHAEVYVQTHGSKVANLPEGQYRYADGTLTRFSDDIVLKKQVIAINQSVYQAASFGISVISRAPEEWMHYVTLGKKLQHLMMNGLGLGFMSSGYSSKTGNPLPASRRIDSVLQANGVESGPSYFFVGGRVSDEQLGHEGMREDSVHMRGPAELIRDDLVSFLPDYMIPNRVVVFERLPLSANGKIDAKALAASDQVNAELVERPFVAPRTETEKEIAEVWAKSLRRESVSVQDDFFESGGNSLIAVGLIRELNSRLGVSLPLQSVLESPTVEKLSRRLEREVAQESSRLVRLHAETGKDRPVLCWPGLGGYPMNLRTLAGEIGLGRSFYGIQAHGINEGEAPYATITEMAKADIEAIKELQPKGPYTLWGYSFGARVAFETAYQLEQAGEKVDNLFLIAPGSPTVRAENGKVYGREASFANRAYTTILFSVFTGTISGPDLEKCLESATDEESFAGFISELKGIDVDLAKRIISVVGQTYEFEYSFRELAERTLAAPVTIFKARGDDYSFIENSNGYSAEPPTVIDLDADHYSLLRTPDIGELVKHIRYLLGE

SEQ ID NO: 5 - protein, 1377 aa, Streptomyces chromofuscus
MSVETIPCSRRAALGLPGLLRERARATPDRTAAVHEHQSLTFAQLTEDSSHVGALLRQAGVGRDSRVGVFMEPSLDLLTGVWGILWAGGCYVPLSPEYPEERIAYMLADAGVDIVLTQEFLRSTLQELAPAGVVVLTLDEMLRTAERDGSAFGRPEPEVRPDDLAYVIYTSGSTGKPKGVMVEHRSIVSQMRWLHDECGIDENEIILQKTPMSFDAAQWELLALACGSTVVMGSSGIYRDPEAIISTVQRHGVTTLQCVPTLLQALLDTEKFPDCGTLRRIFSGGEALSRSLAAQCLDTMPGARLVNLYGPTECTINASSFVVDRAALEDGPLVMPIGTPVHDTSLHVLRPDGAPVSAGEIGELYIGGVQVARGYLGRPDLTGDRFMADPFSDAPGSRLYRTGDLAHVNADGTVQFVGRADNQVKLRGYRVELDEIRQTVETHDWVRAAAVLLRDDATTGFQNLVAFVELNPKEAALMDQGNHGSHHQSKASRLQVRAQLAHPGCRDDADLAGRAAIDLPGAEATPGQRALAFSRKTYRFYEGSPVTRDDILHLLGPRPRPRPSARTSDIVGRDELGTILRNFGRHLSDQRLLPKYAYASPGSLYATQLYVEIGGGHDVPAGLYYYHPLHHRLVLVGPASETETSPVRIHFLGKHGAIEPVYRNNVREVLEIEAGHMVGLFEEVLPAHGLRIAAAAYQPAVRHRLDCAPEDHYLGSFDLLPQARGASEDTDTLDIYVQAHSTRIEGLPPGQYRYTGAGLVRIGDDVILKKHVIAINQRVYERSDFGISLVATGSASWRRYLDLGRGLQRLQMNDLHLGFMSSGYSSKSGNDLPSAKRLGRILADGGLPAGPSYFCVGGRVSDAQWRGEDMKEDVVHMQGPAELIKEDLAALLPRYMLPNRIVVLDRLPQTANGKIDLKALQTTQEAQLTVGERAFMAPRTPLERRIRDIWQAVLKRDQVSVTDDFFELGGNSLLAVALVSRLNADFGGAIPLQILFEAPTVERLAAALEATSPRPASRLVPLQPEGRGTPLYCWPGLGGYPMNLRPLAAALGTERPVHGVQAHGINPGEFPYDDVRAMAAADVEAIREIQPHGPYLLCGYSFGARVAFEAARQLEQAGEQVEQLFLVAPGQPRLRPEDAVGATGRADFTDRAFLALLFSVFAGTLSGPRLDQCLRTVTDEDGFVAFVTASFPGLGEELVRAVTGIVRRTYSLTYEFHELRGRRLDAPVTLVRATDDNYSFIEHEGGYSARPPAVHQLRSGHYELLREPHVARLAAVLNDRLSAGPSTSPRHSQPAQATVQEVGVPHINIKHFPVSITEEKELELVAAVTTAVRNAFGCTEEVVSIALEPVAQEVWNERVYIPEIVARQELLRKTPNY